(19) United States
(12) Patent Application Publication
(10) Pub. No.: US 2007/0065422 A1
(43) Pub. Date: Mar. 22, 2007

(54) Induction of immune tolerance by sertoli cells

(76) Inventors: Don F. Cameron, Lutz, FL (US); Nagwa El-Badri Dajani, Seminole, FL (US); Rania Shamekh, Tampa, FL (US); Samuel Saporta, Tampa, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/524,003

(22) Filed: Sep. 20, 2006

Related U.S. Application Data
(60) Provisional application No. 60/718,648, filed on Sep. 20, 2005.

(51) Int. Cl. A61K 48/00

(52) U.S. Cl. 424/93.21; 424/93.7

(57) ABSTRACT

The subject invention pertains to methods to enhance the therapeutic effects of cell therapy in various diseases and disorders. More particularly, the present invention provides methods of inducing systemic immune tolerance, thereby reducing or eliminating the need for systemic immunosuppression, by administering isolated Sertoli cells to an individual in need of treatment, wherein the isolated Sertoli cells are administered to the individual prior to or during administration of the therapy, e.g., cellular therapy.

### CROSS-REFERENCE TO RELATED APPLICATION

The present application claims benefit of U.S. Provisional Application Ser. No. 60/718,648, filed Sep. 20, 2005, which is hereby incorporated by reference herein in its entirety, including any figures, tables, nucleic acid sequences, amino acid sequences, and drawings.

### BACKGROUND OF THE INVENTION

Cell transplantation therapy is a potentially powerful tool in the treatment of diseases for which there are currently no practical cures. Theoretically, the replacement of defective cells by healthy cells offers the possibility of alleviating the devastating symptoms for many such diseases including Parkinson's disease, stroke, Alzheimer's disease, spinal cord injury, type I diabetes, cirrhosis of the liver and factor 8 hemophilia. The success of the “Edmonton protocol”, which resulted in a 100% cure rate for human Type I diabetes following the transplantation of islet allografts (Shapiro, A. M. et al. N Engl J Med, 2000, 343:230-238), attests to this attractive potential. Clearly, allo- and xenografted cells can restore function to dysfunctional tissues in experimental animal models of disease (for review see Emerich, D. F. et al. Cell Transplant, 2003, 12:335-349)). However, as long as these protocols require persistent systemic immunosuppression, their practical use as clinically relevant therapeutic protocols is unlikely since most immunosuppressant protocols cause severe side effects incompatible with a normal life style (Gaya, S. B. et al. Transplantation, 1995, 59:1705-1709; London, N.J. et al. Lancet, 1995, 346:403-406).

In general, systemic immunosuppression is necessary if successful transplantation is to be achieved in humans. Immunosuppression of the entire body (i.e. systemic) can result, eventually, in graft acceptance. It is acquired, however, by placing the individual at medical risk making the immunosuppressant therapy itself more of a liability than a benefit in some cases. For a lack of a better immunosuppressant treatment, systemic immunosuppressants, with Cyclosporine-A (CsA) as the treatment choice, have been used as adjunctive therapy in neural transplantation protocols. Arguably, systemic CsA treatment may be contraproductive to successful graft acceptance in the CNS because of its systemic effect and because CsA itself has been shown to cause detrimental side effects and may, in fact, be cytotoxic to neural tissues.

Recently, studies have suggested that Sertoli cells, when simultaneously transplanted with pancreatic islet cells into the diabetic rat, act as an effective local immunosuppressant on the host tissue (Selawry, H. P. and Cameron, D. F. Cell Transplant, 1993, 2:123-129). As a result, the graft is not rejected and the islets remain viable allowing the transplanted β-cells to function normally and produce insulin for an indefinite period of time. As a result, the accepted graft overcomes the primary physiological dysfunction of hyperglycemia thereby alleviating the related complications of this endocrine disorder. It is clear that the efficacy of the treatment is due to the presence of the Sertoli cells, in part, due to their known immunosuppressive secretory factor (Selawry, H. P. and Cameron, D. F. Cell Transplant, 1993, 2:123-129; Cameron, D. F. et al. Transplantation, 1990, 50:649-653). Sertoli cells are also known to secrete a number of important trophic growth factors. This cell transplantation protocol is accomplished without prolonged systemic immunosuppression, otherwise necessary when islets are transplanted without Sertoli cells.

Sertoli cells are a permanent population of cells found in mammalian testes which, in the adult, are terminally differentiated. They provide a dynamic trophic factor-rich microenvironment for developing spermatids in a sequestered testicular compartment devoid of blood and lymphatic vasculature, and play an essential role in preventing the individuals from rejecting their own highly antigenic mature germ cells (Pollanen, P. and Niemi, M. International Journal of Andrology, 1987, 10:37-42). The mechanism by which Sertoli cells impart immunoprotection to the mature germ cells was thought to be the consequence of an elaborate network of Sertoli-Sertoli junctional complexes, the so-called “blood-testis barrier”, that physically sequestered these cells from the systemic immune system. It is now known, however, that antigenic germ cells reside outside of the “blood-testis barrier” but avoid rejection, undoubtedly by Sertoli cell secretory factors that immunoprotect these non-sequestered germ cells (Yule, T. D. et al. J Immunol, 1988, 141:1161-1167).

On this basis and following the early observations of Selawry and Cameron (Selawry, H. P. and Cameron, D. F. Cell Transplant, 1993, 2:123-129; Selawr y, H. P. et al. Transplantation, 1991, 52:846-850) who showed that isolated Sertoli cells can create a testis-like immune privileged site outside of the testis, extra-testicular Sertoli cells have been utilized in a number of cell allo- and xenograft cell transplantation protocols (for review see (Emerich, D. F. et al. Cell Transplant, 2003, 12:335-349; Halberstadt, C. et al. Expert Opin Biol Ther, 2004, 4:813-825). The two most studied areas for the transplantation application of extra-testicular Sertoli cells have been with experimental models of diabetes (Korbutt, G. S. et al. Diabetes, 1997, 46:317-322; Korbutt, G. S. et al. Diabetologia, 2000, 43:474-480; Selawry, H. P. and Cameron, D. F. Cell Transplant, 1993, 2:123-129; Shapiro, A. M. et al. N Engl J Med, 2000, 343:230-238; Suarez-Pinzon, W. et al. Diabetes, 2000, 49:1810-1818; Wright, J. R., Jr. and Pohajdak, B. Cell Transplant, 2001, 10:125-143; Yang, H. et al. Cell Transplant, 2002, 11:799-801) and neurodegenerative diseases of the central nervous system (Borlongan, C. V. et al. Exp Neurol, 1997, 148:388-392; Cameron, D. F. “Formation and structure of transplantable tissue constructs generated in simulated microgravity from Sertoli cells and neuron precursors” Cell Transplant, 2004, (in Press); Emerich, D. F. et al. Cell Transplant, 2003, 12:335-349; Othberg, A. I. et al. Cell Transplant, 1998, 7:157-164; Rodriguez, A. I. et al. Neurotox Res, 2002, 4:103-109; Rodriguez, A.I. et al. Neurotox Res., 2003, 5:443-450; Sanberg, P. R. et al. Nat Med, 1997a, 3:1129-1132; Sanberg, P. R. et al. Nat Biotechnol, 1996, 14:1692-1695; Sanberg, P. R. et al. Transplant Proc., 1997b, 29:1926-1928; Sanberg, P. R. et al. Neurotox Res., 2002, 4:95-101; Saporta, S. et al. Exp Neurol., 1997, 146:299-304; Saporta, S. et al. Brain Res Bull, 2004, 64:347-356; Willing, A. E. et al Mol Med Today, 1998, 4:471-477; Willing, A. E. et al. Brain Res, 1999a, 822:246-250; Willing, A. E. et al. Brain Res Bull, 1999b, 48:441-444). It is clear from these and other reports that the co-transplantation of extra-testicular Sertoli cells provide substantial trophic support to cells and tissue grafts, and also reduce the likelihood of both allo- and xenograft rejection even in the absence of systemic immunosuppression (Halberstadt, C. et al. Expert Opin Biol Ther, 2004, 4:813-825; Saporta, S. et al. Exp Neurol., 1997, 146:299-304). It is not yet clear, however, how extra-testicular Sertoli cells immunoprotect cell and tissue grafts, although a number of theories have been offered (Emerich, D. F. et al. Cell Transplant, 2003, 12:335-349; Halberstadt, C. et al. Expert Opin Biol Ther, 2004, 4:813-825; Willing, A. E. et al. Mol Med Today, 1998, 4:471-477; Willing, A. E. et al. Brain Res, 1999a, 822:246-250; Willing, A. E. et al. Brain Res Bull, 1999b, 48:441-444). In general, the ability of extra-testicular Sertoli cells to cause a significant reduction or even elimination of allo- or xenograft rejection appears to be related to their close proximity to the co-transplanted cells and tissues, since most of the transplantation protocols using extra-testicular Sertoli cells have been performed by co-transplantation or co-encapsulation of the cells. This has prompted the suggestion that Sertoli cells provide local immunosuppression at the graft site (Cameron, D. F. “Formation and structure of transplantable tissue constructs generated in simulated microgravity from Sertoli cells and neuron precursors” Cell Transplant, 2004, (in Press); Cameron, D. F. et al. In Vitro Cell Dev Biol Anim, 2001a, 37:490-498; Cameron, D. F. et al. Ann N Y Acad Sci, 2001b, 944:420-428; Emerich, D. F. et al. Cell Transplant, 2003, 12:335-349; Halberstadt, C. et al. Expert Opin Biol Ther, 2004, 4:813-825; Korbutt, G. S. et al. Diabetes, 1997, 46:317-322; Korbutt, G. S. et al. Diabetologia, 2000, 43:474-480; Sanberg, P. R. et al. Nat Biotechnol, 1996, 14:1692-1695; Selawry, H. P. and Cameron, D. F. Cell Transplant, 1993, 2:123-129; Willing, A. E. et al. Mol Med Today, 1998, 4:471-477; Willing, A. E. et al. Brain Res, 1999a, 822:246-250; Yang, H. et al. Cell Transplant, 2002, 11:799-801; Yang, H. and Wright, J. R., Jr. Transplantation, 1999, 67:815-820).

The possibility, however, that extra-testicular Sertoli cells may impart their “immunosuppressive” or “immunoprotective” properties by a mechanism other than local devices was first presented by Bellgrau and Selawry (Bellgrau, D. and Selawry, H. P. Transplantation, 1990, 50:654-657). They showed that when rat cryptorchid testes were successful transplanted with hamster islet xenografts, subsequent hamster islet transplantations either beneath the kidney capsule or in the liver of the host rat resulted in successful islet engraftment. Control rats, who did not first receive hamster islet xenografts in their cryptorchid testes, rejected the hamster islet transplants (Bellgrau, D. and Selawry, H. P. Transplantation, 1990, 50:654-657). They suggested, from this work, that Sertoli cells may induce some type of systemic tolerance in the host. However, this hypothesis of systemic immune modulation has been challenged. Korbutt and co-workers tested induction of immune tolerance by transplanting islet allografts and allogenic Sertoli cells under the rat kidney capsule (Korbutt, G. S. et al. Diabetes, 1997, 46:317-322). This was followed by removal of the grafted kidney and the additional transplantation of rat islets in the contralateral kidney. If systemic tolerance had been induced by the initial transplant, then the subsequent islet transplants should have been accepted. This was not the case. Instead, the secondary transplants resulted in a hyperimmune response and islet rejection. Since then, little research has been directed toward elucidating the effects of transplanted extra-testicular Sertoli cells on the systemic immune system and the mechanism(s) by which these testis-derived cells result in immunoprotection of co-transplanted cells and tissues remains unclear.

### BRIEF SUMMARY OF THE INVENTION

The present invention concerns a method of inducing systemic immune tolerance for transplanted cells in a subject by administering isolated Sertoli cells prior to or during transplantation of donor cells. The method of the invention reduces or eliminates the need for systemic immunosuppression.

### BRIEF DESCRIPTION OF DRAWINGS

FIG. 1 shows mixed lymphocytic response in rSC-and PTC-transplanted C57/BL6 mice 6 weeks post-transplantation. Spleen cells were collected from rSC- and PTC-transplanted mice and non-transplanted controls and cultured with irradiated cells from self, allogenic, syngenic and xenogenic cells to asses tolerance. Self: spleen cells from the same mouse. Stimulator: irradiated spleen cells from transplanted, non-transplanted mice, rat spleens, rSC and PTC. Responder: spleen cells from transplanted and non-transplanted mice. Results were reported as stimulator index (mean cpm of experimental culture divided by mean cpm of culture with autologus cells)±SEM. *p<0.05,

FIG. 2 shows IgM response in C57/BL6 mice that were immunized with SRBCs 5 days prior to collection of spleen cells. Primary anti-sheep red blood cell (SRBC) IgM response was determined by the number of plaques/10⁵ cells. (A) Non-transplanted mice; (B) rSC-transplanted mice and (C) PTC-transplanted mice. Results were reported as means+ SEM.

FIG. 3 shows results of flow cytometric analysis to identify spleen T-cell phenotype. Spleen cells from rSC- and PTC-transplanted animal (2×10⁵ cells), and non-transplanted animals were collected 8 weeks post-transplant. The selected population was based on total percentage of CD 45 positive cells. Results were reported as mean+SEM.

FIG. 4 shows results of flow cytometric analysis to identify thymus T-cell phenotype. Thymus cells from rSC- and PTC-transplanted animal (2×10⁵ cells), and non-transplanted animals were collected 8 weeks post-transplant. The selected population was based on total percentage of CD 45 positive cells. Results were reported as mean±SEM.

FIG. 5 shows cytokine expression in plasma of rSC (n=4) and PTC (n=3) transplanted mice, and non-transplanted B6 (n=2) control mice 6 weeks post-transplant. Data are expressed as (means+SEM). *p<0.05 and **p<0.01 compared to both PTC- and non-transplanted control mice.

FIG. 6 shows post-transplantation rat skin graft survival in C57/BL6 recipient mice. (A) iSC-transplanted mice received rat skin graft 1 mo after iSC injection (n=6). (B) non-transplanted control mice receiving skin graft (n=4). P<0.05.

FIGS. 7A-7D show rat skin grafts on C57/BL6 mice. FIG. 7A shows a non-transplanted C57/BL6 control mouse receiving C57/BL6 skin. FIG. 7B shows non-transplanted C57/BL6 control mice receiving rat skin 8 days after skin grafting. FIG. 7C shows rSC-transplanted C57/BL6 experimental mice receiving rat skin 12 days after skin grafting. FIG. 7D shows rSC-transplanted C57/BL6 experimental mice receiving rat skin 20 days after skin grafting.

### DETAILED DISCLOSURE OF THE INVENTION

Cell therapy is a potentially powerful tool in the treatment of many grave disorders including leukemia, immune deficiencies, autoimmune diseases and diabetes. However, finding matched donors is challenging and recipients may suffer from the severe complications of systemic immune suppression. Sertoli cells, when co-transplanted with both allo- and xenograft tissues, promote graft acceptance in the absence of systemic immunosuppression. The present inventors have examined the ability of Sertoli cells to produce systemic immunotolerance. For this purpose, rat Sertoli cells (rSC) were injected into an otherwise normal C57/BL6 mouse host via the lateral tail vein. No other immunosuppressive protocols were applied. Six to eight weeks post-transplantation, blood was collected for analysis of cytokine levels, thymus and spleen cells were analyzed by flow cytometry. Tolerance to donor cells was determined by mixed lymphocytic cultures, and production of T-cell dependant antibody was determined by in vitro anti-sheep red blood cell plaque forming assay. Tolerance was also determined by grafting rat skin onto mice that had been “tolerized” by the prior IV injection of rat Sertoli cells into the mouse. Results showed a marked modulation of immune cytokines in the transplanted mice and donor specific transplantation tolerance was achieved. Tolerant lymphocytes maintained a competent humoral antibody response. In addition, tolerized mice (i.e. mice that had been injected with rat Sertoli cells) showed acceptance of the rat skin as demonstrated by the significant increased graft survival time which was twice as long as that observed in mice that had not been “tolerized” with the rat Sertoli cells. The present inventors have demonstrated that systemic administration of rat Sertoli cells across the xenogenic barrier induces transplantation tolerance without altering systemic immune competence. This data suggest that Sertoli cells could be used as a novel and potentially powerful tool in xenogenic cell transplantation therapy.

The Sertoli cells can be administered to a subject to treat or prevent disorders associated with an abnormal or unwanted immune response associated with cell, tissue or organ transplantation, e.g., renal, hepatic, and cardiac transplantation, e.g., graft versus host disease (GVHD), or to prevent allograft rejection.

In some embodiments, a therapeutically effective amount of Sertoli cells can be, e.g., the amount necessary to reduce T cell proliferation by about at least 20%. In some embodiments, T cell proliferation is reduced by at least about 30%, about 40%, about 50%, about 60%, about 70% about 80%, or about 90% compared to levels in the absence of Sertoli cell treatment. In some embodiments, a therapeutically effective amount of Sertoli cells is the amount necessary to decrease levels of Th1 cytokines (such as IL-1 beta, IL-2, IL-6, and/or TGF-alpha), and/or IL-10 as measured in the peripheral blood by about 20% or more. In some embodiments, levels of one or more of these cytokines is reduced by at least about 30%, about 40%, about 50%, about 60%, about 70% about 80%, or about 90% compared to levels in the absence of Sertoli cell treatment. Concentrations of IL-1 beta, IL-2, IL-6, IL-10, and/or TGF-alpha, or levels of cells secreting these cytokines, can be measured in the peripheral blood, e.g., using an enzyme-linked immunosorbent assay (ELISA) or a cell-based assay such as FACS scanning, to monitor the induction of tolerance.

Optionally, the Sertoli cells are administered concurrently with one or more second therapeutic modalities, e.g., symptomatic treatment, high dose immunosuppressive therapy. Such methods are known in the art and can include administration of agents useful for treating an autoimmune disorder, e.g., NSAIDs (including selective COX-2 inhibitors); other antibodies, e.g., anti-cytokine antibodies, e.g., antibodies to IFN-alpha, IFN-gamma, and/or TNF-alpha; heat shock proteins (e.g., as described in U.S. Pat. No. 6,007,821); immunosuppressive drugs (such as corticosteroids, e.g., prednisolone and methyl prednisolone; cyclophosphamide; azathioprine; mycophenolate mofetil (MMF); cyclosporin and tacrolimus; methotrexate; or cotrimoxazole) and therapeutic cell preparations, e.g., subject-specific cell therapy. In a preferred embodiment, no immunosuppressive is administered to the subject; thus, only the immune tolerance of the Sertoli cells is relied upon to prevent or delay onset of transplant rejection.

The methods described herein can also be used to treat or prevent graft rejection in a transplant recipient. For example, the methods can be used in a wide variety of tissue and organ transplant procedures, e.g., the methods can be used to induce central tolerance in a recipient of a graft of cells, e.g., stem cells such as bone marrow and/or of a tissue or organ such as pancreatic islets, liver, kidney, heart, lung, skin, muscle, neuronal tissue, stomach, and intestines. Thus, the new methods can be applied in treatments of diseases or conditions that entail cell, tissue or organ transplantation (e.g., liver transplantation to treat hypercholesterolemia, transplantation of muscle cells to treat muscular dystrophy, or transplantation of neuronal tissue to treat Huntington's disease or Parkinson's disease). In some embodiments, the methods include administering to a subject in need of treatment: a) an effective amount of Sertoli cells (systemically administered); and b) donor cells. The donor cells can be isolated cells or comprise tissue or an organ, e.g., liver, kidney, heart, lung, skin, muscle, neuronal tissue, stomach and intestines.

In some embodiments, the transplanted cells comprise pancreatic islets. Accordingly, the invention encompasses a method for treating diabetes by pancreatic islet cell transplantation. The method comprises administering to a subject in need of treatment: a) an effective amount of Sertoli cells (systemically administered); and b) donor pancreatic islet cells. Typically, the Sertoli cells are administered to the recipient prior to and/or simultaneously with administration of the pancreatic islets.

In some embodiments, the recipient is then treated with a regimen of immune-suppressing drugs to further suppress rejection of the donor cells (e.g., isolated cells, tissue, or organ). Standard regimens of immunosuppressive treatment are known. Tolerance to donor antigen can be evaluated by standard methods, e.g., by MLR assays.

In some embodiments, the donor is a living, viable human being, e.g., a volunteer donor, e.g., a relative of the recipient. In some embodiments, the donor is no longer living, or is brain dead, e.g., has no brain stem activity. In some embodiments, the donor tissue or organ is cryopreserved. In some embodiments, the donor is one or more non-human mammals, e.g., an inbred or transgenic pig, or a non-human primate.

Mammalian species which benefit from the methods of the invention include, but are not limited to, primates, such as apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. As used herein, the terms “patient”, “individual”, “subject”, “host”, and “recipient” are interchangeable and intended to include such human and non-human mammalian species.

Typically, the dose of donor cells administered to a patient, particularly a human, in the context of the present invention should be sufficient to achieve a therapeutic response in the patient over a reasonable time frame. One skilled in the art will recognize that dosage will depend upon a variety of factors including the condition of the animal, the body weight of the animal, as well as the severity and stage of the cancer.

Patients in need of treatment using the methods of the present invention can be identified using standard techniques known to those in the medical profession.

The donor cells can be administered as cell therapy to alleviate the symptoms of a wide variety of disease states and pathological conditions, in various stages of pathological development. For example, donor cells can be used to treat acute disorders (e.g., stroke or myocardial infarction), and administered acutely, subacutely, or in the chronic state. Similarly, the donor cells can be used to treat chronic disorders (e.g., Parkinson's disease, diabetes, or muscular dystrophy), and administered preventatively and/or prophylactically, early in the disease state, in moderate disease states, or in severe disease states. For example, the donor cells can be administered to a target site or sites on or within a patient in order to replace or compensate for the patient's own damaged, lost, or otherwise dysfunctional cells. This includes infusion of the cells into the patient's bloodstream. The cells to be administered can be cells of the same cell type as those damaged, lost, or otherwise dysfunctional, or a different cell type. For example, insulin-producing pancreatic islet beta cells supplemented with other types of cells of the subject invention can be administered to the liver (Goss, J. A., et al., Transplantation, Dec. 27, 2002, 74(12):1761-1766). As used herein, patients “in need” of the donor cells include those desiring elective surgery, such as elective cosmetic surgery.

The Sertoli cells and donor cells can be administered as autografts, syngeneic grafts, allografts, and xenografts, for example. The Sertoli cells and donor cells administered to the subject may be obtained from any of the aforementioned species in which the cells are found. As used herein, the term “graft” refers to one or more cells intended for implantation within a human or non-human subject. Hence, the graft can be a cellular or tissue graft, for example.

The Sertoli cells and donor cells can be administered to a patient by any method of delivery, such as intravascularly, intracranially, intracerebrally, intramuscularly, intradermally, intravenously, intraocularly, orally, nasally, topically, or by open surgical procedure, depending upon the anatomical site or sites to which the cells are to be delivered. Donor cells can be administered in an open manner, as in the heart during open heart surgery, or in the brain during stereotactic surgery, or by intravascular interventional methods using catheters going to the blood supply of the specific organs, or by interventional methods such as intrahepatic artery injection of pancreatic cells for diabetics. Preferably, the immune tolerance inducing Sertoli cells are administered systemically, such as intravenously.

The pharmaceutical compositions used in the method of the invention can be formulated according to known methods for preparing pharmaceutically useful compositions. Formulations are described in a number of sources which are well known and readily available to those skilled in the art. For example, Remington's Pharmaceutical Sciences (Martin EW, 1995, Easton Pa., Mack Publishing Company, 19^{th} ed.) describes formulations which can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. It should be understood that in addition to the ingredients particularly mentioned above, the formulations of the subject invention can include other agents conventional in the art having regard to the type of formulation in question.

The Sertoli cells and/or donor cells may also be administered intravenously or intraperitoneally by infusion or injection. Solutions of the cells can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the Sertoli cells or donor cells in the desired amount in the appropriate solvent with any of the other various ingredients enumerated above, as required, followed by filter sterilization.

As used herein, the terms “administering,” “introducing,” “delivering,” “placement” and “transplanting” are used interchangeably herein and refer to the placement of cells into a subject by any method or route that results in at least partial localization of the cells at a desired site. Thus, “transplanted” cells include those that have been grown in vitro, and may have been genetically modified, as well as the transplantation of material extracted from another organism. The cells can be administered by any appropriate route which results in delivery to a desired location in the subject where at least a portion of the cells or components of the cells remain viable. The period of viability of the cells after administration to a subject can be as short as a few hours, e.g., twenty-four hours, to a few days, to as long as several years.

As used herein, the term “treating” includes reducing or alleviating at least one adverse effect or symptom of a disease or disorder.

As used herein, “therapeutically effective dose of cells” refers to an amount of cells that are sufficient to bring about a beneficial or desired clinical effect. The dose could be administered in one or more administrations. However, the precise determination of what would be considered an effective dose may be based on factors individual to each patient, including, but not limited to, the patient's age, size, type or extent of disease, stage of the disease, route of administration of the cells, the type or extent of supplemental therapy used, ongoing disease process and type of treatment desired (e.g., aggressive vs. conventional treatment).

The terms “recombinant host cells”, “host cells”, “cells”, “cell lines”, and other such terms denoting microorganisms or higher eukaryotic cell lines refer to cells which can be, or have been, used as recipients for recombinant vectors or other transfer DNA, immaterial of the method by which the DNA is introduced into the cell or the subsequent disposition of the cell. The terms include the progeny of the original cell that has been transfected. The Sertoli cells and donor cells can be those of primary cultures, or cells which have been passaged one or more times, for example. In a preferred embodiment, the Sertoli cells and donor cells are cells of cell lines.

The Sertoli cells, donor cells, or both may be genetically modified or non-genetically modified cells. The term “genetic modification” as used herein refers to the stable or transient alteration of the genotype of a cell by intentional introduction of exogenous nucleic acids by any means known in the art (including for example, direct transmission of a polynucleotide sequence from a cell or virus particle, transmission of infective virus particles, and transmission by any known polynucleotide-bearing substance) resulting in a permanent or temporary alteration of genotype. The nucleic acids may be synthetic, or naturally derived, and may contain genes, portions of genes, or other useful polynucleotides. A translation initiation codon can be inserted as necessary, making methionine the first amino acid in the sequence.

The Sertoli cells, donor cells, or both may be transformed or non-transformed cells. The terms “transfection” and “transformation” are used interchangeably herein to refer to the insertion of an exogenous polynucleotide into a host cell, irrespective of the method used for the insertion, the molecular form of the polynucleotide that is inserted, or the nature of the cell (e.g., prokaryotic or eukaryotic). The insertion of a polynucleotide per se and the insertion of a plasmid or vector comprised of the exogenous polynucleotide are included. The exogenous polynucleotide may be directly transcribed and translated by the cell, maintained as a nonintegrated vector, for example, a plasmid, or alternatively, may be stably integrated into the host genome.

Preferably, the donor cells administered to the subject are non-Sertoli cells. The donor cells can range in plasticity from totipotent or pluripotent stem cells (e.g., adult or embryonic), precursor or progenitor cells, to highly specialized or mature cells, such as those of the central nervous system (e.g., neurons and glia). Stem cells can be obtained from a variety of sources, including fetal tissue, adult tissue, umbilical cord blood, peripheral blood, bone marrow, and brain, for example. Methods and markers commonly used to identify stem cells and to characterize differentiated cell types are described in the scientific literature (e.g., Stem Cells: Scientific Progress and Future Research Directions, Appendix E1-E5, report prepared by the National Institutes of Health, June, 2001). The list of adult tissues reported to contain stem cells is growing and includes bone marrow, peripheral blood, umbilical cord blood, brain, spinal cord, dental pulp, blood vessels, skeletal muscle, epithelia of the skin and digestive system, cornea, retina, liver, and pancreas. As will be understood by one of skill in the art, there are over 200 cell types in the human body. It is believed that the methods of the subject invention can be used to induce immune tolerance in a subject for transplants comprising any of these cell types for therapeutic or other purposes. For example, any cell arising from the ectoderm, mesoderm, or endoderm germ cell layers can be administered under the immunotolerance provided by the Sertoli cells. Examples of cell types that can be administered using the methods of the subject invention include those disclosed by Spier R. E. et al., eds., (2000) The Encyclopedia of Cell Technology, John Wiley & Sons, Inc., and Alberts B. et al., eds., (1994) Molecular Biology of the Cell, 3^{rd} ed., Garland Publishing, Inc., e.g., pages 1188-1189, each of which are incorporated herein by reference in their entirety.

It should be understood that the Sertoli cells can be administered to a subject to induce immune tolerance for donor cells, regardless of the purposes the donor cells were administered. Thus, the method of the invention may also be utilized when it is desired to transplant cells for non-therapeutic purposes (i.e., for purposes other than cell therapy). For example, the method of the invention can be used to provide immune tolerance for transplanted cells in a subject, wherein the transplanted cells were administered to the subject for research purposes. In this case, the transplanted donor cells need not provide any therapeutic effect. Such research purposes include, but are not limited to, the study of cell migration and differentiation, and the cellular decisions that occur during cell determination and differentiation. Furthermore, the ability of the transplanted cells to express endogenous or heterologous genes in a human or non-human subject in vivo can be studied.

The Sertoli cells and/or donor cells can include one or more labels. For example, the donor cells can be labeled to track their migration and/or differentiation within the subject's tissue in vivo or ex vivo (see, for example, Cahill, K. et al. Transplantation, 2004, 78(11):1626-1633; and Lekic, P. C. et al. Anat Rec, 2001, 262(2):193-202; Bulte, J. W. et al. gEuro Cells and Mater, 2002, 3(2):7-8; Turnbull, D. H. et al. Proc Intl Soc Mag Reson Med, 2001, 9:359; Dunning, M. D. et al. J Neurosci, 2004, 24(44):9799-810; and Kaufman, C. L. et al. Transplantation, 2003, 76(7):1043-1046).

The terms “comprising”, “consisting of” and “consisting essentially of” are defined according to their standard meaning. The terms may be substituted for one another throughout the instant application in order to attach the specific meaning associated with each term.

The terms “isolated” or “biologically pure” refer to material that is substantially or essentially free from components which normally accompany the material as it is found in its native state. Thus, isolated Sertoli cells in accordance with the invention preferably do not contain materials normally associated with the Sertoli cells in their in situ environment.

As used in this specification, the singular forms “a”, “an”, and “the” include plural reference unless the context clearly dictates otherwise. Thus, for example, a reference to “a cell” includes more than one such cell (e.g., can include tissue and organs) or type of cell. A reference to “a cytokine” includes more than one such cytokine, and so forth.

The practice of the present invention can employ, unless otherwise indicated, conventional techniques of molecular biology, microbiology, recombinant DNA technology, electrophysiology, and pharmacology that are within the skill of the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover Ed. 1985); Perbal, B., A Practical Guide to Molecular Cloning (1984); the series, Methods In Enzymology (S. Colowick and N. Kaplan Eds., Academic Press, Inc.); Transcription and Translation (Hames et al. Eds. 1984); Gene Transfer Vectors For Mammalian Cells (J. H. Miller et al. Eds. (1987) Cold Spring Harbor Laboratory, Cold Spring Harbor, N.Y.); Scopes, Protein Purification: Principles and Practice (2nd ed., Springer-Verlag); and PCR: A Practical Approach (McPherson et al. Eds. (1991) IRL Press)), each of which are incorporated herein by reference in their entirety.

Any feature or combination of features described herein are included within the scope of the present invention provided that the features included in any such combination are not mutually inconsistent as will be apparent from the context, this specification, and the knowledge of one of ordinary skill in the art.

Materials and Methods

Animals: Forty 6-8 week old C57/BL6 male mice and 8 Balb/c male mice (Harlan Laboratories and Jackson Laboratories), weighing 20-25 g at the time of surgery, were used in these experiments. Prepubertal Sprague-Dawley rats were used to isolate Sertoli cells (rSC) and peritubular cells (PTC), and to harvest normal spleens. All animals were routinely housed with free access to food and water in a temperature controlled room on a 12:12 h light cycle. At 6-8 weeks following cell transplantation, experimental and control mice were euthanized using CO₂ inhalation.

Cell preparation: Testes from 15-17 day old male Sprague Dawley rats (Harlan Laboratories) were used to isolate both Sertoli cells (rSC) and peritubular cells (PTC). The tunica albuginea was removed from the individual testes, the testicular parenchyma pooled, and then subjected to sequential enzymatic digestion with tryspin (0.25%) and collagenase (0.2%) at 37° C. as previously described (Cameron, D. F. and Muffly, K. E. J Cell Sci, 1991, 100(Pt 3):623-633). Prior to the isolation of Sertoli cell aggregates, the supernatant following collagenase treatment was collected and centrifuged to isolate PTC. Prior to plating, all cells were washed three times with DMEM (Gibco, BRL).

Sertoli cells aggregates were plated in 150 mm cell culture dishes in DMEM:F12 (Gibco, BRL) supplemented with 0.1% ITS+(Sigma), 50 ng/ml retinol acetate (Sigma) and 50 μμg/ml gentamicin sulfate (Sigma), and incubated at 39° C. in 5% CO₂₋95% air for 48 hours to expedite the removal of contaminating germ cells. These cultures then were exposed to 20 mM Tris-HCl buffer for 2 minutes to lyse remaining germ cells, washed and returned to the CO₂-injected incubator at 37° C. in 5% CO₂−95% air. Using this procedure, pre-treated rat Sertoli cell-enriched cultures (rSC) contained greater that 95% SC.

Peritubular cells (PTC), collected during the isolation procedure, were plated in 150 mm cell culture dishes in DMEM:F12 supplemented with 10% fetal bovine serum (FBS, Gibco, BRL) and 50 μg/ml gentamicin sulfate and incubated at 37° C. in 5% CO₂−95% air until confluent.

Cell transplantation: C57/BL6 (HO2b) mice were administered cell transplants in a volume of 0.5 ml sterile medium by intravenous injection in the lateral tail vein. Recipient mice received either suspended rSC in the concentrations of 2×10⁶ (n=8) and 4×10⁵ (n=8) or PTC also in the concentrations 2×10⁶ (n=8) and 4×10⁵ (n=8).

Mixed lymphocytic reaction (MLR): Spleens were harvested from animals transplanted with rSC (n=4) and PTC (n=4) (1×10⁶). As well as normal C57/BL6 BALB/C mice and Sprague Dawely rats and were used as controls. MLR was performed as previously described (El Badri, N. S. and Good, R. A. Proc Soc Exp Biol Med, 1994, 205:67-74). Briefly, responder spleen cells (2×10⁵/100 μl) from transplanted mice were co-cultured with irradiated (2500 rads) stimulator spleen cell (2×10⁵/100 μl) in 96-well flat bottom plates. These cells also were co-cultured in RPMI 1640 medium with 10% FBS, 1% penicillin-streptomycin (Sigma) for 4-days in humidified 5% CO₂ at 37° C. After which they were incubated for 18 hours with 1 μCi of [³H] thymidine per well, harvested on glass fiber papers and [³H] thymidine uptake assessed with a scintillation counter. Results were expressed as a stimulation index, calculated as the mean of counts (3 replicates) per minute (cpm) of the experimental co-culture divided by mean cpm of co-culture with autologous cells. Mann-Whitney U-Test was used for non-parametric analysis. Significance level was set at p<0.05.

Plague forming cell (PFC) response to in vivo immunization with sheep red blood cells (SRBC): C57/BL6 mice (n=4) transplanted with 1×10⁶ rSC, or 1×10⁶ PTC (n=4), and C57/BL6 non-transplanted mice were used for this assay (Jeme, N. K. and Nordin, A. A. Science, 1963, 140:405). Briefly, mice were immunized by intraperitoneal injection of 0.5 ml of 2% suspension of freshly collected sheep red blood cells (SRBC) in 1× PBS (phosphate buffer saline, Fisher Biotech. BP661-10, Fisher Scientific, Fair Lawn, N.J.). Five days after immunization, mice were euthanized and spleen cells collected for PFC.

Flow cytometric analysis: C57/BL6 mice (n=3) transplanted with 1×10⁶ rSCs, or transplanted with 1×10⁶ PTC (n=3) and C57/BL6 non-transplanted mice were used for this analysis. Transplanted mice were euthanized 8 weeks post-transplantation. Spleens and thymuses were collected; single cell suspensions were prepared, washed, counted and evaluated for viability using trypan blue dye exclusion. Likewise, spleen and thymus cells were prepared from control age and sex matched C57/BL6 control non-transplanted mice. Cells were collected into 5 ml v-bottom tubes (VWR International #160818-096) and spun for 5 minutes at room temperature at 350×g. The pellet was washed once with chilled staining buffer (1×PBS with 0.1% w/v bovine serum albumin [BSA Sigma A-3294, St. ouis Mo.] and 0.01% [w/v] sodium azide [NaN₃, Fisher Biotec. #BP922-500]). Spleen and thymus cells were placed in staining buffer for 5 minutes and spun 5 minutes at room temperature at 350×g. The pellet was washed and resuspended in 50 μl of buffer for every 5×10⁴ cells. Monoclonal antibodies to mouse surface cell antigens were used at a concentration of 0.2 to 0.5 μg/0.1 ml staining buffer. Antibodies used were anti-CD45-PERCP (BD-P cat. #557235), anti-CD4-PE (BD-P cat. #557308), anti-CD8-FITC (BD-P cat. #553040), anti-CD28-PE (BD-P cat. #553297), and CD40-FITC (BD-P cat. #553723). Cells were incubated for 30 minutes at 4° C., in v-bottom 5 ml tubes, washed once, and then fixed with 0.3 ml freshly prepared 1% (v/v) paraformaldehyde solution in 1×PBS from a 16% stock solution (Electron Microscopy Sciences cat. #15710). Flow acquisition and analysis was performed on a FACSCaliburrm™, (BD Biosciences).

Cytokines: Blood (0.5-1 ml) was collected in heparinized syringes from C57/BL6 transplanted with either rSC or PTC (1×10⁶ cells) or normal controls. Blood samples were stored on ice for 30 min, spun down, plasma collected and stored at −80° C. Protein array for IL-1α, IL-1β, IL-2, IL-4, IL-6, IL-10, IL-12p70, TNF-α and GMCSF was done using SearchLight CCD Imaging and Analysis System (Pierce). Results were expressed as mean+ standard error of the mean (SEM). Analysis of variance was used to determine differences in mean. Significance level was set at p<0.05.

Rat-to-mouse skin graft: For the xenogenic skin transplantation experiment, iSCs were prepared from prepubertal rats as described above and injected (1×10⁶) into male C57/BL6 mice (n=6) also as described in the in vitro studies. Four C57/BL6 mice did not receive iSC injection and served as controls. The donor skin was shaved and a full thickness circular piece of skin (diameter=1 cm) was removed from the naive rat (Sprague-Dawley) under sterile conditions after euthanasia with CO₂ gas. A similar circular piece of host skin was removed from the C57/BL6 mouse and the donor skin was fitted within the wound on the back of the host mouse. The donor rat skin was sutured in place using 4-0 or 5-0 non absorbable suture (Prolene) using a simple interrupted pattern. Bandages were not needed. The grafts were observed daily for 4 weeks. Graft rejection was defined when>50% of the graft showed signs of inflammations (redness, tenderness, and exudation) and scabbing. Results were expressed as mean± standard error of the mean (SEM). Analysis of variance was used to determine differences in the mean. Significance level was set at p<0.05.

Example-Immuno-Tolerization of Mice to Rat Tissue Graft Using Rat Sertoli Cells

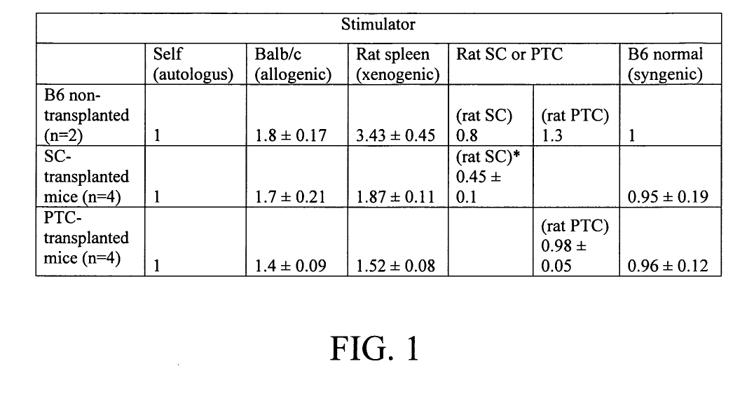

Animals were allowed to survive up to 6 months post-transplantation. All animals were healthy at the time of sacrifice with no signs of systemic rejection. rSC-transplanted mice are tolerant to xenogenic cells: A mixed lymphocytic reaction assay was used to detect tolerance induced by rSC or PTC (FIG. 1). Spleen cells obtained from rSC-transplanted C57/BL6 mice were stimulated with irradiated MHC-mismatched allogeneic Balb/c, xenogenic rat spleen cells, rat rSC, or syngenic C57/BL6 spleen cells (self). There was significant decrease in the recipient spleen T-lymphocyte response (p<0.05) to xenogenic rat rSC (0.45±0.1) compared to control non-transplanted mice (0.8). There was also a decrease in the recipient spleen T-lymphocyte response to rat spleen cells (1.87±0.11) compared to control non-transplanted (3.43±0.45), but variability between groups prevented these differences from reaching significance. Response to allogeneic Balb/c spleen cells (1.7±0.21) was intact and comparable to control non-transplanted mice (1.8±0.17). Spleen cells from PTC-transplanted mice were also less reactive to rat spleen cells (1.52±0.08) but maintained this low reactivity to allogeneic Balb/c stimulation (1.4±0.09).

Anti-sheep erythrocyte (SRBC) plaque forming cell assay (PFA) in C57/BL6 transplanted and non-transplanted mice: Production of humoral immune response was assayed using PFA assay, which determines primary antibody (IgM) response to SRBC (T-cell dependant antigen). Plaque formation appears as a clear zone (plaque) representing an 1 gM producing plasma cell in the presence of complement. The number of plaques was individually counted using a magnifying lens. C57/BL6 mice transplanted with (1×10⁶) xenogenic rat rSC (n=3) (FIG. 1) showed an increase in IgM anti-SRBC antibody response (44.1±5.93) compared to response in non-transplanted C57/BL6 (n=2) mice (29.4±8.13). This increase was not specific to rSC, since a comparable increase in anti-SRBC IgM response was similarly observed in mice transplanted with xenogeneic rat PTC (42.11±8.13).

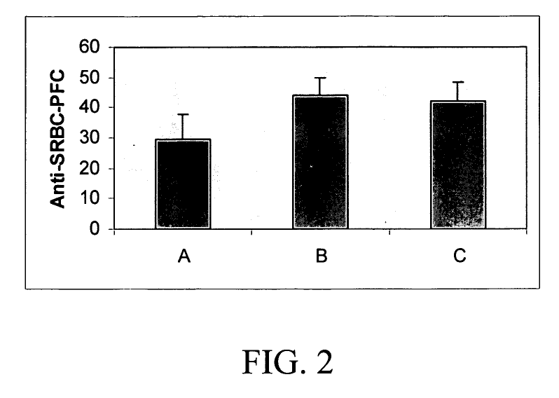

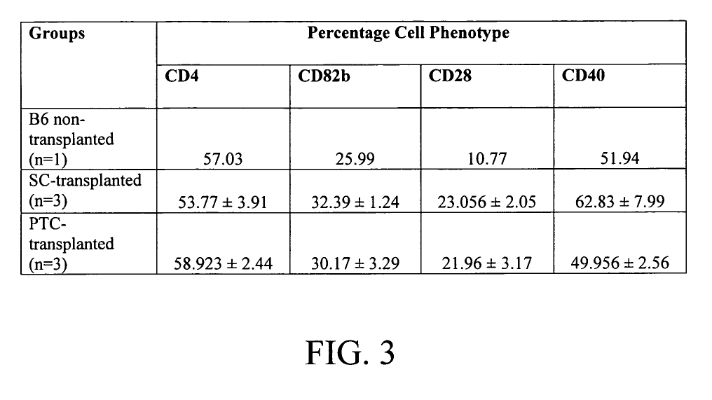

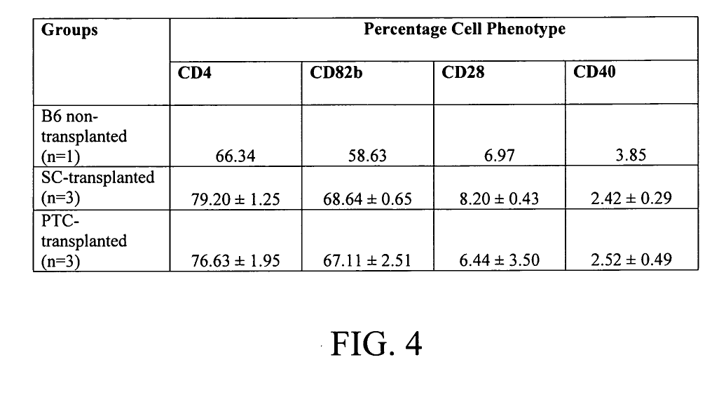

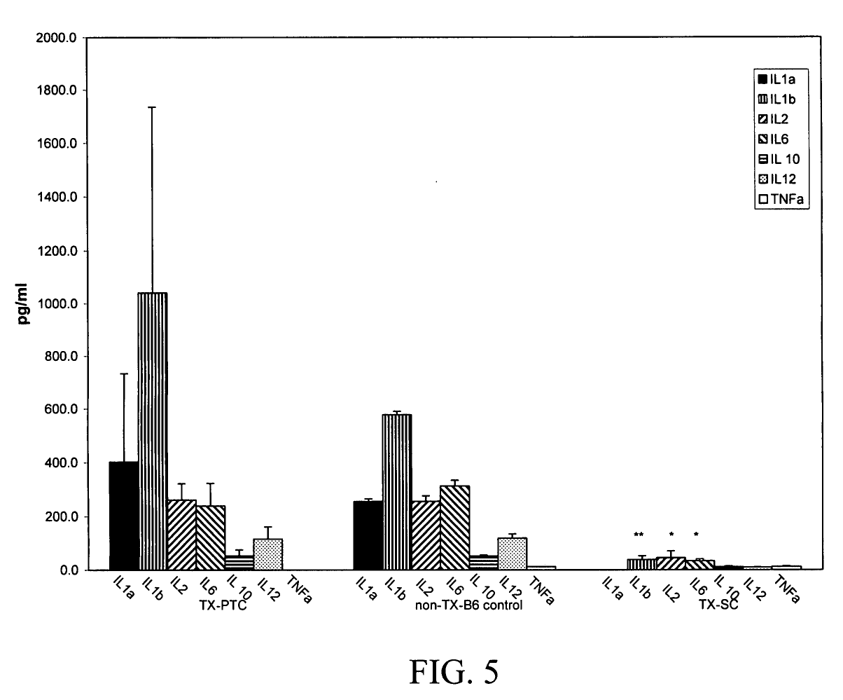

Flow cytometric analysis of SC and PTC-transplanted animals compared to non-transplanted normal controls (FIGS. 2 and 3). To determine the effect of Sertoli cell transplantation on T-lymphocyte profile, both spleen and thymus cells from transplanted and control non-transplanted C57/BL6 mice were examined for expression of CD4 helper and CD8 cytotoxic/suppressor phenotype. The relative expression of CD28 and CD40 antigens, which are important in tolerance induction and maintenance (FIG. 2), were also analyzed. Spleen lymphocytes harvested from C57/BL6 mice transplanted with xenogenic rat rSC showed a change in CD8, CD28 and CD40 compared to non-transplanted control mice However, no change was observed in CD4 phenotype in C57/BL6 controls and rSC-transplanted mice. Spleen cells from C57/BL6 mice transplanted with xenogenic rat PTC showed marked expression of CD28, but slight change in CD8 phenotype. There was minimal change in the CD4 and CD40 populations.

Unlike splenic lymphocytes, thymus T-cell profile from rSC-transplanted mice showed change in both CD4 and CD8 lymphocyte populations and respectively compared to non-transplanted C57/BL6 mouse CD4 and CD8, (FIG. 3). A slight change in CD40 and CD28 expression was detected in rSC-transplanted mice compared to C57/BL6 non-transplanted CD40 and CD28. Interestingly, thymus cells of PTC-transplanted mice showed minimal change in the CD4, CD8, CD40 cell populations, and no change in the CD28 thymus population.

Cytokine analysis of SC- and PTC-transplanted mice compared to non-transplanted normal controls (FIG. 2). Plasma from rSC (n=4), PTC (n=3) transplanted and non-transplanted (n=2) normal mice was examined for cytokine expression 6 weeks post-transplant. The cytokine profile included IL-1α, IL-1β, IL-2, IL-4, IL-6, IL-10, IL-12p70, TNF-α and GM-CSF, measured in pg/ml. Remarkably, plasma from rSC-transplanted mice showed striking decrease in expression of most of the tested cytokines with the exception of TNF-α expression (12.26±1.67) in rSC-transplanted mice compared to non-transplanted control (12.13). The rSC-transplanted mice showed a significant statistical difference when analyzed with ANOVA between treatment groups (F=7.89, df=2, p=0.0014). Newman-Keuls post-hoc analysis showed a significant decrease in IL-1β (38.03±14.71; p<0.01) and IL-2 (44.76±26.67; p<0.05) in rSC mice, compared to either IL-1β (577.14±13.29) and IL-2 (256.18±20.75) in non-transplanted control mice, or to IL-1β (1040.61±694.81) and IL-2 (261.43±60.44) in PTC-transplanted mice. IL-6 was significantly decreased in rSC-transplanted mice (34.16±6.31; p<0.05) compared to non-transplanted mice (312.70±21.37). There was no significant change in IL-10 and IL-12 in the rSC-transplanted mice compared to either non-transplanted control mice or to PTC-transplanted mice. There was no significant difference between PTC-transplanted mice and non-transplanted mice for any cytokine. IL-1α was not detected in the rSC-transplanted mice. It was unclear whether this was due to a technical error, or if IL-1α was suppressed below the limit of detectability in mice receiving rSC. Therefore, no comparison was done between groups for IL-1α. Similarly, levels of IL-4 and GM-CSF were below the limit of detectability and are not reported.

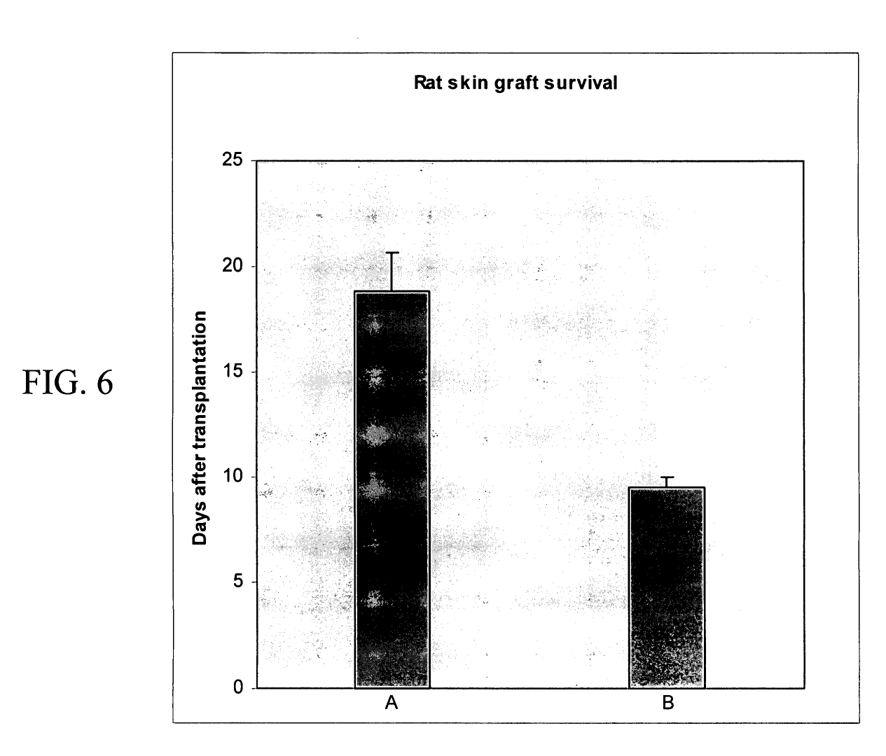

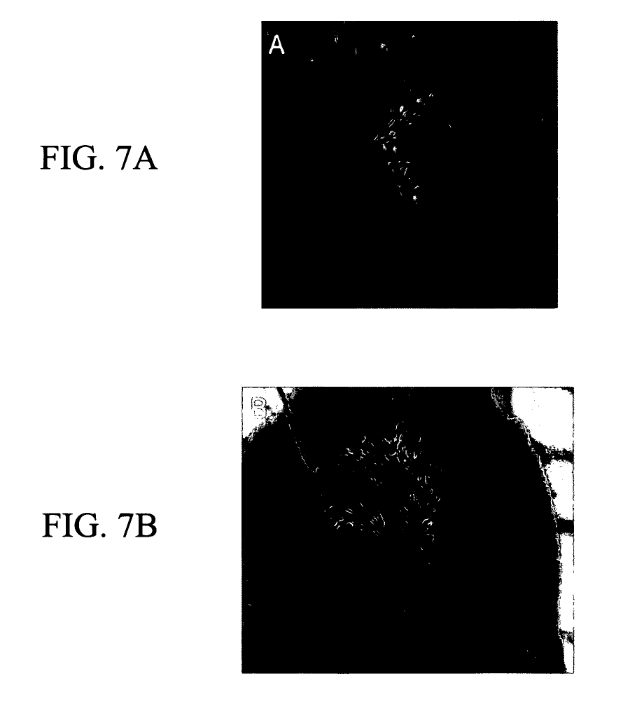

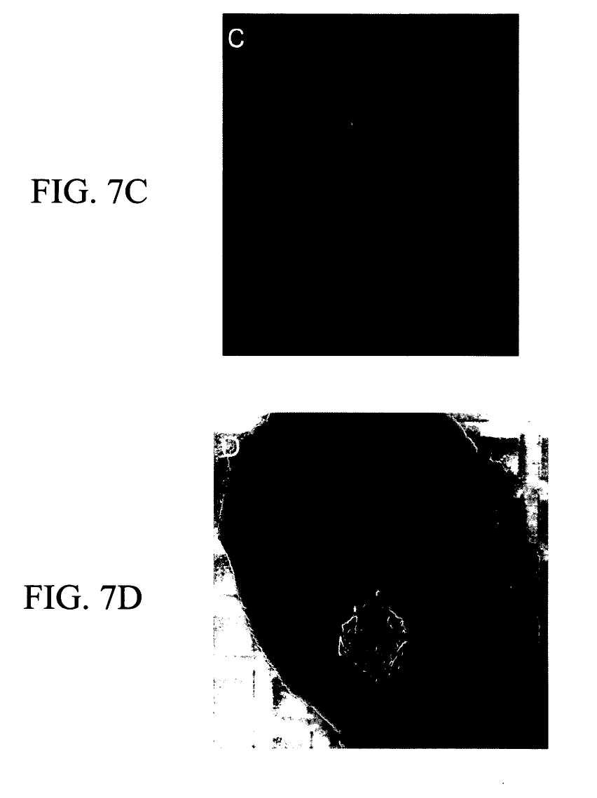

The rat-to-mouse skin graft experiment was performed as an in vivo assessment for rSC-induced immune tolerance. As shown in FIGS. 7A-7D, non-transplanted mice rejected xenogenic rat skin grafts by 9.5±0.5 days. However, the rejection of xenogenic rat skin grafted onto rSC-transplanted mice (i.e. tolerized mice) was significantly delayed by 18.8±1.79 days (p<0.05). Non-transplanted mice showed signs of graft rejection 8 days after skin grafting (FIG. 7B). However, in the iSC-transplanted group, signs of graft rejections were remarkably delayed and ranged between 12 and 23 days after the skin graft (FIGS. 7C and 7D). Non-transplanted C57/BL6 control mice received syngenic C57/BL6 mouse skin as an assessment of skin grafting technique (FIG. 6A) and did not reject the syngenic mouse skin.

Over the past two decades, advances in tissue and organ transplantation have revolutionized the treatment of many otherwise fatal diseases such as aplastic anemia, leukemia, immune deficiencies, inborn errors of metabolism, and organ failures. However, the continued need for immune suppression to ensure long-term durable engraftment has limited the widespread use of allogeneic transplantation. The brain, anterior eye chamber, testes, ovaries and the placenta are primary immune privileged sites that protect transplanted cells from rejection (Streilein, J. W. Science, 1995, 270:1158-1159). It is well known that Sertoli cells (SC) provide immunoprotection to the highly antigenic spermatid population within the testis. They appear, likewise, to provide immunoprotection in pancreatic islets co-transplants in the periphery to treat Diabetes (Dufour, J. M. et al. Immunol Invest, 2003, 32:275-297) and to neuron precursors co-transplanted in the brain to treat neurodegererative diseases (for reviews, see (Emerich, D. F. et al. Cell Transplant, 2003, 12:335-349) and (Halberstadt, C. et al. Expert Opin Biol Ther, 2004, 4:813-825)). Additionally, cell transplant therapy with isolated prepubertal Sertoli cells in the 3-nitropropionic acid (3-NP) model of Huntington's disease has been shown to provide immunoprotective effects similar to those obtained using systemic treatments with non-steroidal anti-inflammatory drugs (Salzberg-Brenhouse, H. C. etal J Pharmacol Exp Ther, 2003, 306:218-228).

It has previously been shown that rat (allogenic) or porcine (xenogenic) Sertoli cells can survive in the rat brain without immune suppression (Saporta, S. et al. Exp Neurol., 1997, 146:299-304). In the current study, the present inventors show the first evidence for the capacity of isolated Sertoli cells to produce durable systemic tolerance without the need for immune suppression. Rat Sertoli cells isolated from the prepubertal rats were transplanted, by systemic injection, into normal C57/BL6 mice without prior conditioning by immune suppression. Testicular PTC, also isolated from the prepubertal rat, were transplanted as well into the normal C57/BL6 mice as control since these cells are a common, but minimal, contaminant in the isolated SC preparation. Therefore, PTC were ideal to determine if the effect of SC on immune system was solely a characteristic of SC, or if PTC, also obtained from the testis, shared this characteristic. As shown in FIG. 1, spleen lymphocytes from C57/BL6 mice that were transplanted with rat Sertoli cells were specifically tolerant to rat cells (both spleen and rat Sertoli cells) in a mixed lymphocyte reaction. This tolerance was specific for donor rat cells since these C57/BL6 cells were properly allorecative to third party (Balb/c mouse) spleen cells. To confirm that T-lymphocyte tolerance induced by intravenous injection of Sertoli cells was not associated with generalized immune suppression, lymphocytes from transplanted mice were tested for their capacity to induce a humoral immune response to a T-cell dependent antigen, sheep red blood cells (SRBCs) (El Badri, N. S. and Good, R. A. Proc Soc Exp Biol Med, 1994, 205:67-74). Spleen lymphocytes from mice transplanted with Sertoli cells produced vigorous primary antibody response to immunization with SRBCs FIG. 1. These data confirm that mice transplanted with pre-pubertal rat Sertoli cells are immune competent and capable of eliciting proper immune reaction to foreign antigens, while specifically tolerant of xenogenic donor-specific antigens.

Pre-transplantation conditioning is responsible for the majority of early fatalities in transplant patients in the form of infection and graft versus host disease (GVHD), produced by immune competent T-lymphocytes within the graft (Baden, L. and Rubin, R. H. “Infection in hematopoietic stem cell transplant recipients” Soiffer, R. A., ed. In: Stem Cell Transplantation for Hematologic Malignancies. Totowa, N. J.: Humana Press, Inc., 2004:237-258). When Sertoli cells were transplanted in large numbers (1×10⁶ cells), crossing the rat-mouse xenogenic barrier, no immune suppression was required prior to transplantation. The mice were healthy and showed no signs of GVHD, which is the main complication of other forms of toleralizing cell therapy regimens such as bone marrow transplantation (Ferrara, J. L. et al. Biol Blood Marrow Transplant, 1999, 5:347-356; Parkman, R. Curr Opin Hematol, 1998, 5:22-25).

In bone marrow transplant chimeras, donor T-cell production was critical for maintenance of long-term transplantation tolerance (Xu, H. et al. J. Immunol, 2004,172:1463-1471). In the experiments described herein, the durable tolerance produced by Sertoli cells for 8 weeks post-transplantation was independent of donor T-lymphocytes. This suggests an alternative mechanism for tolerance induction that may involve cytokines and soluble mediators. In transplantation models, Th1 cytokine profile is usually associated with allograft rejection, while Th2 cytokine profile is associated with transplantation tolerance. However, recent data suggest that this paradigm may be reversed where in vivo administration of Th2 cytokines stimulated allograft rejection (Zhai, Y. et al. Crit Rev Immunol, 1999, 19:155-172). The cytokine profile expression shown in FIG. 2 demonstrates a marked suppression in the inflammatory Th1 cytokine profile (IL-1, IL-2, IL-6 and TNF-α), as well as the suppression of some Th2 regulatory cytokines (IL-10). Additional analysis of some key Th2 regulators like IL-4 is required. Nonetheless, the significant suppression of various inflammatory cytokines suggests that the hCD4 population, especially the Th1 subpopulation, is dramatically modulated by intravenous administration of Sertoli cells isolated from the prepubertal rat.

To further investigate this hypothesis, the phenotype of thymic lymphocytes was analyzed before and after transplantation. CD4⁺ population was minimally reduced in the spleen but significantly elevated in the thymus of mice transplanted with SC. This elevation was not Sertoli cell-specific however, since a similar increase was observed in thymuses of mice transplanted with rat PTC, suggesting a cross-species xenogeneic reaction. Similar data were observed with the CD8⁺ cell population. The dissociation between the suppressed inflammatory cytokine profile and the increased and functional sub-populations of T-lymphocytes suggests that tolerance induced by SC is independent of T-lymphocyte production, but related to T-lymphocyte stimulation. This hypothesis is supported by the observed increase in the CD8 population, which indicates that a population of suppressor T-cells may also contribute to the observed tolerance.

Activation of T-lymphocytes involves co-stimulation of the B7:CD28 and the CD40 ligand (CD40L):CD40 pathway. TCR transgeneic mice lacking CD28 showed defective Th2 and a defective proliferative response (Howland, K. C. et aL J. Immunol, 2000, 164:4465-4470). In SC transplanted mice, CD28 expression was intact in thymus cells and increased in spleen cells. This increase, however, seems related to xenogeneic transplantation since a similar increase was obtained in cells from mice transplanted with rat PTC. A similar observation was obtained with CD40 expression on spleen and thymus cells FIGS. 2 and 3. Larsen and co-workers (Larsen, C. P. et al. Nature, 1996, 381:434-438) reported that simultaneous, but not independent, blockade of the CD28 and CD40 pathways effectively inhibited T-cell clonal expansion in vitro and in vivo, and promoted long-term survival of fully allogeneic skin and cardiac allografts. The intact CD28 and CD40 expression in the SC transplanted mice suggests that SC mediated tolerance is independent of blocking B7:CD28 and the CD40L:CD40 T-cell activation pathways.

Transplantation tolerance induced by other forms of cell therapy, such as bone marrow transplantation, promotes desirable acceptance of donor tissues and organ grafts. For example, transplantation tolerance in rat recipients of intestinal allografts was improved by co-transplantation of donor strain marrow cells (Nakao, A. et al. Transplantation, 2003, 75:1575-1581). Donor specific tolerance for allogeneic hepatocytes was achieved in mice transplanted with allogeneic marrow cells (Yoshida, N. et al. Hepatol Res, 2003, 26:148-153). Sertoli cells have been shown to produce tolerance at the site of injection since GFP-expressing Sertoli cells transplanted under the kidney capsule of SCID and Balb/c mice promoted islet engraftment at both 30 and 60 days post-transplantation (Dufour, J. M. et al. Gene Ther, 2004, 11:694-700).

To determine whether the immune tolerizing effect of systemic administration of rSCs could be extended to an in vivo situation, rat skin grafts were transplanted onto naive mice and onto mice transplanted with rSCs. Delayed rejection of the skin grafts in the rSC-transplanted groups confirms that the immune tolerance achieved by systemic administration of rSCs may have clinical applications, and allows acceptance of other organs and tissue grafts. In this study, xenogenic skin graft rejection was delayed in some cases by almost three weeks. Although this is not a permanent type of tolerance, since eventually all skin grafts were rejected, systemic administration of rSCs could be considered highly promising when compared to other tolerizing forms of cell therapy like bone marrow transplantation (Baden, L. and Rubin, R. H. “Infection in hematopoietic stem cell transplant recipients” Soiffer, R. A., ed. In: Stem Cell Transplantation for Hematologic Malignancies. Totowa, N. J.: Humana Press, Inc., 2004:237-258). Achieving long lasting durable tolerance may necessitate manipulating factors like modifying the dose of rSCs, or repeated injections of rSCs.

The systemic effects of Sertoli cells, as shown by the experiments described herein, indicate that the novel delivery of rSCs by systemic injection induce tolerance to donor-type cells, in the host mouse, which is consistent with a similar conclusion by Bellgrau and coworkers (Bellgrau, D. and Selawry, H. P. Transplantation, 1990, 50:654-657) but inconsistent with a report to the contrary by Korbuttt and coworkers (Korbutt, G. S. et al. Diabetologia, 2000, 43:474-480). The latter authors suggested that SCs could not induce tolerance to transplanted islets in the diabetic rat. It is difficult to compare and contrast, however, the results of the experiments described herein to those of Bellgrau and Korbutt in that the SCs in their projects were not transplanted by systemic injection nor were assays of systemic immune modulation reported, as is the case in the current report. It is clear that co-transplantation of islets and SCs under the kidney capsule did not allow for immune acceptance of islets transplanted in the contralateral kidney (Korbutt, G. S. et al. Diabetologia, 2000, 43:474-480) indicating the absence of induced tolerance by the initial graft. It is possible, however, that the number of transplanted rSCs and their isolation in the kidney (i.e. not systemically injected as in the experiments described herein) did not provide for the extent of systemic exposure to induce tolerization by SCs as was the case in the experiments described herein, and that the absence of islet rejection was the result of local immunoprotection by SCs, as suggested by others. In this sense, the results described herein may not be in conflict with those of Korbutt and co-workers (Korbutt, G. S. et al. Diabetologia, 2000, 43:474-480), but may instead indicate that immunoprotective properties attributed to “local” or “graft site” dependant properties of co-transplanted SCs are the result of a systemic effects adequate to immunoprotect the graft not sufficient to induce tolerance. In the Belgrau et al. study (Bellgrau, D. and Selawry, H. P. Transplantation, 1990, 50:654-657), Sertoli cells were not isolated for systemic or local delivery (i.e. they were not transplanted), but were in situ SCs resident in the host's own testes. They did not test for systemic immune modulation to account for the acceptance of the islet graft in the kidney following islet transplantation in the testis, and their speculation that Sertoli cells may induce “some sort of tolerance” may have been the result of some sort of local immunoprotective effects by Sertoli cells independent of the cells' ability to induce tolerance when isolated and injected into another animal as demonstrated by the experiments described herein.

The effects of transplanting isolated Sertoli cells (by systemic delivery) on the systemic immune system, as described herein, indicates that the tolerizing effects of isolated Sertoli cells could be applied to various organs and tissue grafts as well as other cell transplantation therapies.

The invention has been described in an illustrative manner, and it is to be understood the terminology used is intended to be in the nature of description rather than of limitation. Obviously, many modifications and variations of the present invention are possible in light of the above teachings and one of ordinary skill in the art, in, light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

All patents, patent applications, provisional applications, and publications referred to or cited herein are incorporated by reference in their entirety, including all figures and tables, to the extent they are not inconsistent with the explicit teachings of this specification.

We claim:

1. A method for inducing systemic immunotolerance in a subject, comprising administering an effective amount of isolated Sertoli cells to the subject.

2. The method of claim 1, wherein donor cells are administered to the subject prior to, or during, said administering of the Sertoli cells, and wherein the donor cells are not Sertoli cells.

3. The method of claim 1, wherein the Sertoli cells are administered to the subject systemically.

4. The method of claim 2, wherein the Sertoli cells are administered to the subject systemically.

5. The method of claim 4, wherein the Sertoli cells are administered to the subject intravenously.

6. The method of claim 2, wherein the donor cells are allogenic to the subject.

7. The method of claim 2, wherein the donor cells are xenogenic to the subject.

8. The method of claim 1, wherein systemic immune competence is maintained in the subject.

9. The method of claim 2, wherein systemic immune competence is maintained in the subject.

10. The method of claim 1, wherein no immunosuppressive agents are administered to the subject.

11. The method of claim 2, wherein no immunosuppressive agents are administered to the subject.

12. The method of claim 2, wherein the donor cells are administered to the subject as cell therapy, to replace or compensate for the subject's own damaged, lost, or dysfumctional cells.

13. The method of claim 2, wherein the donor cells are stem cells.

14. The method of claim 2, wherein the donor cells are pancreatic islet cells and the subject is suffering from diabetes.

15. The method of claim 2, wherein the donor cells are administered in the form of a tissue or organ graft.

16. The method of claim 2, wherein the donor cells are genetically modified cells.

17. The method of claim 1, wherein the Sertoli cells are administered multiple times.

18. The method of claim 1, wherein the subject is human.

19. The method of claim 1, wherein the Sertoli cells are administered to the subject in an amount effective to reduce T cell proliferation, the concentration of Th1 cytokines, or the concentration of IL-10 in the peripheral blood of the subject.

20. A method for delaying the onset of immune rejection to allogenic or xenogenic donor cells in a subject, comprising systemically administering an effective amount of isolated Sertoli cells to the subject prior to, or during, administration of the donor cells to the subject.
